(19) United States
(12) Patent Application Publication
(10) Pub. No.: US 2010/0280387 A1
(43) Pub. Date: Nov. 4, 2010

(54) Active Delay Line for Continuous Wave Ultrasound System

(76) Inventor: Rea Richard Schmid, Rochester, MN (US)

Correspondence Address: TEXAS INSTRUMENTS INCORPORATED, P O BOX 655474, M/S 3999, DALLAS, TX, 75265, US

(21) Appl. No.: 12/432,100

(22) Filed: Apr. 29, 2009

(51) Int. Cl. A61B 8/00; G06G 7/12

(52) U.S. Cl. 600/457; 327/355

(57) ABSTRACT

A delay line circuit is provided for aggregating a plurality of CW ultrasound echo signals. The circuit comprises a plurality of series coupled active component delay stages each having a respective tap input configured to receive a respective CW ultrasound echo signal of the plurality of ultrasound echo signals and provide an aggregated ultrasound echo signal.

### TECHNICAL FIELD

This invention relates to ultrasound, and more specifically to an active delay line for a continuous wave ultrasound system.

### BACKGROUND

In performing ultrasonic diagnosis by a continuous wave (CW) Doppler method, a Doppler shift of an echo of continuous wave ultrasound is calculated, and the calculated Doppler shift is displayed as a frequency spectral image or a sound. The frequency spectral image or sound is information representing, for example, the velocity of blood flow. When the direction of echo reception is electronically set by a phased array technique, a phased addition is performed on echoes received by a plurality of ultrasonic transducers in an ultrasonic probe. The phased addition of the received echo signals is performed using a passive component delay line. The passive component delay line has a plurality of input taps provided at different positions in the longitudinal direction of the passive component delay line, and an output tap provided at an end of the passive delay line. A signal input to one of the input taps is output from the output tap with a delay imparted depending upon the tap position. By inputting a plurality of input signals to respective proper input taps depending upon the phase differences among the input signals, all the signals can be put in phase at the output tap. At the output tap, all the inphase signals are superposed to provide a phased added signal of all the input signals. The passive component delay lines are constructed with a series of inductor-capacitor-resistor (LCR) element sections. The tolerance of the discrete components (e.g., inductors) limits the resolution of the ultrasound system and creates numerous problems in amplitude summing and accurate delays.

### SUMMARY

In an aspect of the invention, a continuous wave (CW) ultrasound system is provided. The CW ultrasound system comprises an ultrasonic probe for abutting against a portion of a body of a person, a transmitter that transmits ultrasound transmit signals through the ultrasonic probe and a receiver that receives a plurality of ultrasound echo signals from the ultrasonic probe in response to the ultrasound transmit signals, the receiver comprising an active component delay line formed of a plurality of series coupled active component delay stages each having a respective tap input configured to receive a respective ultrasound echo signal of the plurality of ultrasound echo signals and provide an aggregated ultrasound echo signal.

In another aspect of the invention, a delay line circuit is provided for aggregating a plurality of CW ultrasound echo signals. The circuit comprises a plurality of series coupled active component delay stages each having a respective tap input configured to receive a respective CW ultrasound echo signal of the plurality of ultrasound echo signals and provide an aggregated ultrasound echo signal.

In yet another aspect of the present invention, a method of capturing and displaying an ultrasound image is provided. The method comprises transmitting a plurality of CW ultrasound transmit signals to a portion of a body, receiving a plurality of CW ultrasound echo signals in response to the plurality of CW ultrasound transmit signals and providing the plurality of CW ultrasound echo signals to an active component delay line formed of a plurality of series coupled active component delay stages via a plurality of corresponding tap inputs each associated with a respective delay stage to provide a CW aggregated echo signal. The method further comprises postprocessing the CW aggregated echo signal to provide a postprocessed signal and displaying an ultrasound image on a display based on the postprocessed signal.

### BRIEF DESCRIPTION OF THE DRAWINGS

FIG. 1 illustrates a block diagram of a continuous wave (CW) ultrasound system in accordance with an aspect of the present invention.

FIG. 2 illustrates a block diagram of an active component delay line in accordance with an aspect of the invention.

FIG. 3 illustrates a schematic diagram of a first active component delay stage in accordance with an aspect of the present invention.

FIG. 4 illustrates a schematic diagram of a subsequent active component delay stage that employs a current source tap input in accordance with an aspect of the invention.

FIG. 5 illustrates a schematic diagram of a subsequent active component delay stage that employs a voltage source tap input in accordance with an aspect of the invention.

FIG. 6 illustrates a schematic diagram of a subsequent active component delay stage that employs a voltage source tap input and a transconductance cell in accordance with an aspect of the invention.

FIG. 7 illustrates an example of a method of capturing an ultrasound image in accordance with an aspect of the present invention.

### DETAILED DESCRIPTION

The present invention relates to an active delay line for a continuous wave (CW) ultrasound system. The active delay line comprises a plurality of series coupled active component delay stages each having a respective tap input configured to receive a respective CW ultrasound echo signal of a plurality of CW ultrasound echo signals and provide a CW aggregated ultrasound echo signal. The use of active components in the delay stage replaces passive components in a conventional passive component delay line, such as inductors that have larger error tolerances. Furthermore, the use of active components provides for buffering or amplifying of the CW ultrasound echo signals mitigating signal loss experienced by a conventional passive component delay lines. Furthermore by eliminating the use of large inductors in the delay line, the active delay line can be readily fabricated on silicon as an integrated circuit.

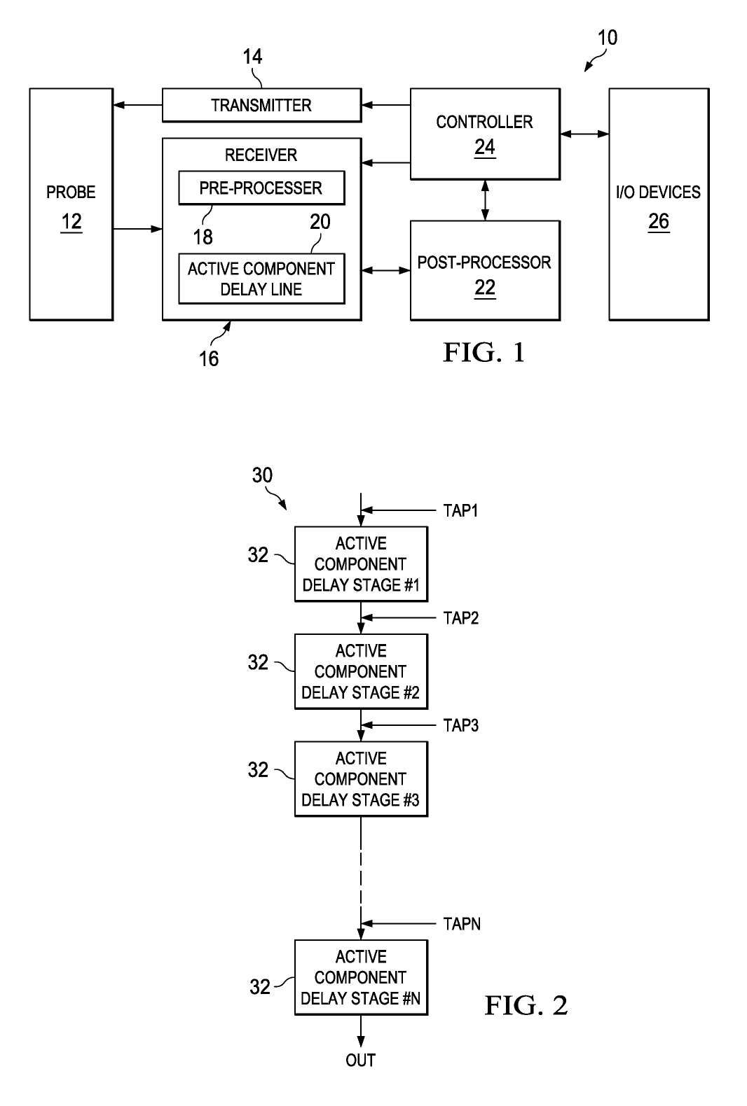

FIG. 1 illustrates a block diagram of a continuous wave (CW) ultrasound system 10 in accordance with an aspect of the present invention. The system 10 has an ultrasonic probe 12 that has an array of a plurality of ultrasonic transducers. The ultrasonic probe 12 can be used by a user by abutting the probe 12 against a portion of a body of a person (not shown). The ultrasonic probe 12 is coupled to a transmitter section 14 and a receiver 16. The transmitter 14 provides a driving signal to the ultrasonic probe 12 to transmit CW ultrasound transmit signals of a predetermined frequency. A plurality of CW ultrasound echo signals is received by the ultrasonic probe 12 in response to the transmitted CW ultrasound transmit signals. The receiver 16 is supplied with individual signals received by the ultrasonic transducers in the ultrasonic probe 12. The multi-channel received CW ultrasound echo signals are amplified and multiplexed by a preprocessor 18 (e.g., an N-channel variable gain amplifier) of the receiver 16 and individually input into an active component delay line 20 of the receiver 16. The outputs of the preprocessor 18 can be in the form of voltage source signals or current source signals. The active component delay line 20 forms a CW aggregated ultrasound echo signal by performing a phased addition on the multi-channel received CW ultrasound echo signals.

The active component delay line 20 is formed of a plurality of active component stages coupled in a series configuration with discrete components (e.g., capacitors, resistors) being employed to set the delay time and gain of the active component stages. The aggregated CW ultrasound echo signals are provided to a postprocessor 22 for performing Doppler processing of the CW aggregated echo signal. A controller 24 receives the processed signal(s) and provides an ultrasound image of the portion of the body to a display of one or more I/O devices 26 based on the processed signal(s). Additionally, sound can be provided to one or more speakers of the one or more I/O devices 26. The I/O devices 26 can also include a keyboard and mouse for inputting information or control signals to the controller 24. The controller 24 can further provide control signals to the transmitter 14, the receiver 16 and/or post processor 22.

FIG. 2 illustrates a block diagram of an active component delay line 30 in accordance with an aspect of the invention. The active component delay line 30 includes a plurality of active component delay stages 32 labeled stage 1-N, where N is an integer greater than one (e.g., 20 delay stages). A respective tap input labeled TAP1-TAPN is provided as input to each corresponding stage. The tap inputs are provided from the preprocessor 18 and provide CW ultrasound echo signals from the probe 12 in the form of a current source input or a voltage source input based on the type of preprocessor being employed. The first active component delay stage of the plurality of stages is configured to set the bias for each of the subsequent active component delay stages. The plurality of active component stages 32 employ discrete components (e.g., resistors, capacitors) to set the delay time and gain of the active component stages. The active component delay line 30 forms a CW aggregated ultrasound echo signal by performing a phased addition on the received multi-channel CW ultrasound echo signals at each tap input.

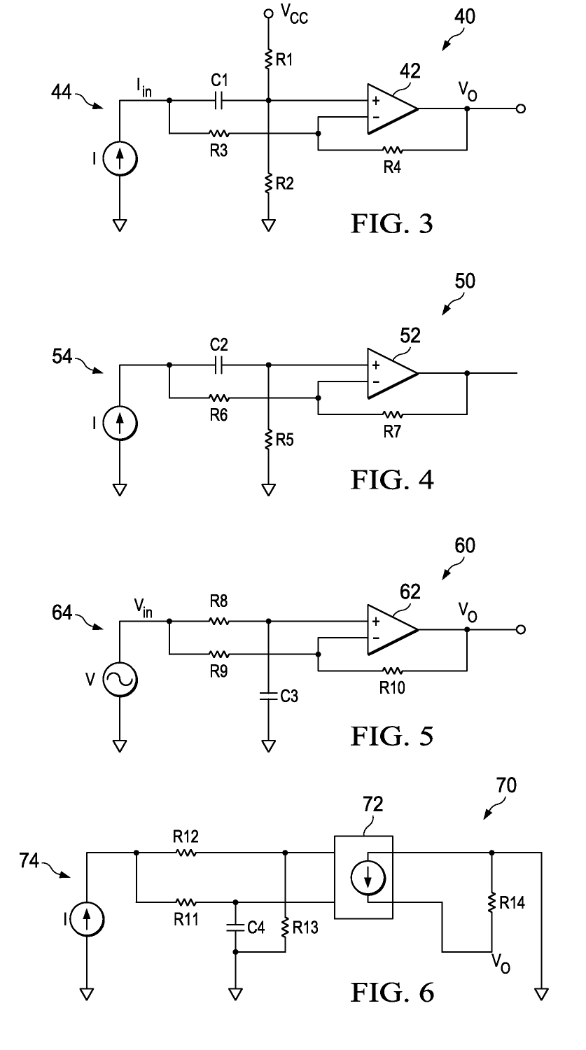

FIG. 3 illustrates a schematic diagram of a first active component delay stage 40 in accordance with an aspect of the present invention. The first active component delay stage 40 sets the bias for the remaining series coupled active component delay stages. In the present example, the first active component delay stage 40 is configured to set the bias of the remaining stages employing a single supply voltage VCC. A first resistor R1 and a second resistor R2 are series coupled between the supply voltage VCC and ground at a first node that is also coupled to a positive input terminal of an operational amplifier 42. The DC voltage at the first node sets the bias for the operational amplifier 42 as well as for the subsequent active component delay stages.

A tap input 44 is provided that is represented as a current source that is coupled to the positive input terminal of the operation amplifier 42 through a capacitor C1 and to a negative input terminal of the operation amplifier 42 through a resistor R3. A resistor R4 is coupled to the negative input terminal of the operational amplifier 42 and an output of the operational amplifier 42. The capacitor C1 and the resistor R1 in parallel with R2 set the delay of the first active component delay stage 40. For example, if R1 and R2 are equal, the DC bias voltage is VCC/2 and the delay would be equal to (R1∥R2)*C excluding the delay of the operational amplifier 42. Furthermore, if the first active component delay stage 40 is to have a same delay as the subsequent stages, R1 and R2 should be about twice the value of delay resistors of the remaining stages. The resistor R4 and R3 set the gain of the first active component delay stage 40.

FIG. 4 illustrates a schematic diagram of a subsequent active component delay stage 50 that employs a current source tap input in accordance with an aspect of the invention. The subsequent active component delay stage 50 is biased by the first active component delay stage 40. It is to be appreciated that the first active component delay stage 40 could be series coupled to a subsequent active component delay stage to form a first stage. A tap input 54 is provided that is represented as a current source that is coupled to the positive input terminal of an operational amplifier 52 through a capacitor C2 and to a negative input terminal of the operational amplifier 52 through a resistor R6. A resistor R5 is coupled between a positive input terminal of the operational amplifier 52 and ground. A resistor R7 is coupled to the negative input terminal of the operational amplifier 52 and an output of the operational amplifier 52. The capacitor C2 and the resistor R5 set the delay of the subsequent active component delay stage 50. The resistor R6 and R7 set the gain of the subsequent active component delay stage 50.

FIG. 5 illustrates a schematic diagram of a subsequent active component delay stage 60 that employs a voltage source tap input in accordance with an aspect of the invention. The subsequent active component delay stage 60 is biased by the first active component delay stage 40. A tap input 64 is provided that is represented as a voltage source that is coupled to the positive input terminal of an operational amplifier 62 through a resistor R8 and to a negative input terminal of the operational amplifier 62 through a resistor R9. A capacitor C3 is coupled between a positive input terminal of the operational amplifier 62 and ground. A resistor R10 is coupled to the negative input terminal of the operational amplifier 62 and an output of the operational amplifier 62. The capacitor C3 and the resistor R8 set the delay of the subsequent active component delay stage 60. The resistor R9 and R10 set the gain of the subsequent active component delay stage 60.

FIG. 6 illustrates a schematic diagram of a subsequent active component delay stage 70 that employs a current source tap input and a transconductance cell in accordance with an aspect of the invention. A transconductance cell provides a current output in response to a voltage input as opposed to an operation amplifier that provides a voltage output in response to a voltage input. A transconductance cell can be readily fabricated on a silicon substrate. The subsequent active component delay stage 70 is biased by the first active component delay stage 40, which could also include a transconductance cell that replaces the operational amplifier 42. A tap input 74 is provided that is represented as a current source that is coupled to a positive input terminal of the transconductance cell 72 through a resistor R12 and to a negative input terminal of the transconductance cell 72 through a resistor R11. A capacitor C4 is coupled between a negative input terminal of the transconductance cell 72 and ground. A resistor R13 is coupled to the positive input terminal of the transconductance cell 72 and ground. The capacitor C4 and the resistor R13 set the delay of the subsequent active component delay stage 70. The resistors R12 and R13 set the gain of the subsequent active component delay stage 70. An output resistor R14 converts the output current into output voltage.

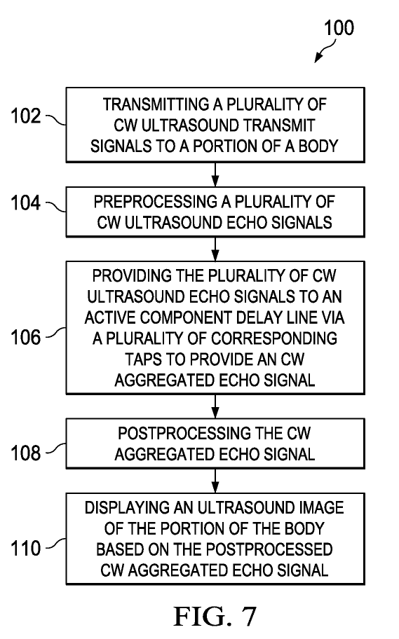

In view of the foregoing structural and functional features described above, certain methods will be better appreciated with reference to FIG. 7. It is to be understood and appreciated that the illustrated actions, in other embodiments, may occur in different orders and/or concurrently with other actions. Moreover, not all illustrated features may be required to implement a method. It is to be further understood that the following methodology can be implemented in hardware (e.g., analog or digital circuitry, such as may be embodied in an application specific integrated circuit), software (e.g., as executable instructions stored in memory or running on a processor implemented in an ASIC), or any combination of hardware and software.

FIG. 7 illustrates an example of a method 100 of capturing an ultrasound image in accordance with an aspect of the present invention. At 102, a plurality of CW ultrasound transmit signals are transmitted to a portion of a body. At 104, a plurality of CW ultrasound echo signals are received and preprocessed (e.g., amplified and multiplexed). At 106, the plurality of CW ultrasound echo signals are provided to an active component delay line via a plurality of corresponding taps to provide a CW aggregated ultrasound echo signal. At 108, the CW aggregated ultrasound echo signal is postprocessed by a Doppler processor. At 110, an ultrasound image is displayed based on the postprocessed CW aggregated ultrasound echo signal.

What have been described above are examples of the invention. It is, of course, not possible to describe every conceivable combination of components or methodologies for purposes of describing the invention, but one of ordinary skill in the art will recognize that many further combinations and permutations of the invention are possible. Accordingly, the invention is intended to embrace all such alterations, modifications, and variations that fall within the scope of this application, including the appended claims.

What is claimed is:

1. A continuous wave (CW) ultrasound system comprising:
an ultrasonic probe for abutting against a portion of a body of a person;
a transmitter that transmits ultrasound transmit signals through the ultrasonic probe; and
a receiver that receives a plurality of ultrasound echo signals from the ultrasonic probe in response to the ultrasound transmit signals, the receiver comprising an active component delay line formed of a plurality of series coupled active component delay stages each having a respective tap input configured to receive a respective ultrasound echo signal of the plurality of ultrasound echo signals and provide an aggregated ultrasound echo signal.

2. The system of claim 1, wherein each of the plurality of series coupled active component delay stages comprises an operational amplifier configured with a resistor-capacitor pair that sets a respective delay time of the active component delay stage and a resistor pair that sets a respective gain of the operational amplifier.

3. The system of claim 1, wherein each of the plurality of series coupled active component delay stages comprises a transconductance cell configured with a resistor-capacitor pair that sets a respective delay time of the active component delay stage and a resistor pair that sets a respective gain of the transconductance cell.

4. The system of claim 1, wherein a first stage of the plurality of series coupled active component delay stages sets a DC bias voltage of each of the plurality of the plurality of series coupled active component delay stages.

5. The system of claim 1, wherein a first stage of the plurality of series coupled active component delay stages comprises a series coupled resistor pair and a capacitor that sets a respective delay time of the first stage and each subsequent stage of the plurality of series coupled active component delay stages comprise a capacitor-resistor pair that sets a respective delay time of the respective stage.

6. The system of claim 5, wherein the series coupled resistor pair is coupled to a supply voltage and sets the DC bias voltage of each of the plurality of series coupled active component delay stages.

7. The system of claim 5, wherein each resistor in the series coupled resistor pair has a value that is about twice the value of the resistor in each of the capacitor-resistor pairs and the capacitors in each of the plurality of series coupled active component delay stages have about the same value such that each of the plurality of series coupled active component delay stages has a substantially equal delay time.

8. The system of claim 1, further comprising a preprocessor that multiplexes and amplifies the plurality of ultrasound echo signals and provides each of the plurality of ultrasound echo signals as one of a voltage source and current source tap input to respective tap inputs of the active component delay line.

9. The system of claim 1, further comprising a postprocessor that Doppler processes the aggregated ultrasound echo signal and provides a postprocessed signal that is employed for displaying an ultrasound image on a display.

10. A delay line circuit for aggregating a plurality of continuous wave (CW) ultrasound echo signals, the circuit comprising:
a plurality of series coupled active component delay stages each having a respective tap input configured to receive a respective CW ultrasound echo signal of the plurality of ultrasound echo signals and provide an aggregated ultrasound echo signal.

11. The circuit of claim 10, wherein each of the plurality of series coupled active component delay stages comprises an operational amplifier configured with a resistor-capacitor pair that sets a respective delay time of the active component delay stage and a resistor pair that sets a respective gain of the operational amplifier.

12. The circuit of claim 10, wherein each of the plurality of series coupled active component delay stages comprises a transconductance cell configured with a resistor-capacitor pair that sets a respective delay time of the active component delay stage and a resistor pair that sets a respective gain of the transconductance cell.

13. The system of claim 10, wherein a first stage of the plurality of series coupled active component delay stages sets a DC bias voltage of each of the plurality of the plurality of series coupled active component delay stages.

14. The system of claim 10, wherein a first stage of the plurality of series coupled active component delay stages comprises a series coupled resistor pair and a capacitor that sets a respective delay time of the first stage and each subsequent stage of the plurality of series coupled active component delay stages comprise a capacitor-resistor pair that sets a respective delay time of the respective stage.

15. The system of claim 14, wherein the series coupled resistor pair is coupled to a supply voltage and sets the DC bias voltage of each of the plurality of series coupled active component delay stages.

16. The system of claim 14, wherein each resistor in the series coupled resistor pair has a value that is about twice the value of the resistor in each of the capacitor-resistor pairs and the capacitors in each of the plurality of series coupled active component delay stages have about the same value such that each of the plurality of series coupled active component delay stages has a substantially equal delay time.

17. A method of capturing and displaying an ultrasound image, the method comprising:
transmitting a plurality of continuous wave (CW) ultrasound transmit signals to a portion of a body;
receiving a plurality of CW ultrasound echo signals in response to the plurality of CW ultrasound transmit signals;
providing the plurality of CW ultrasound echo signals to an active component delay line formed of a plurality of series coupled active component delay stages via a plurality of corresponding tap inputs each associated with a respective delay stage to provide a CW aggregated echo signal;
postprocessing the CW aggregated echo signal to provide a postprocessed signal; and
displaying an ultrasound image on a display based on the postprocessed signal.

18. The method of claim 17, further comprising preprocessing the plurality of CW ultrasound echo signals received in response to the plurality of CW ultrasound transmit signals by amplifying and multiplexing the plurality of CW ultrasound echo signals and providing each of the plurality of ultrasound echo signals as one of a voltage source and current source tap input to respective tap inputs of the active component delay line.

19. The method of claim 17, wherein each of the plurality of series coupled active component delay stages comprises an operational amplifier configured with a resistor-capacitor pair that sets a respective delay time of the active component delay stage and a resistor pair that sets a respective gain of the operational amplifier.

20. The method of claim 17, wherein each of the plurality of series coupled active component delay stages comprises a transconductance cell configured with a resistor-capacitor pair that sets a respective delay time of the active component delay stage and a resistor pair that sets a respective gain of the transconductance cell.
